(19) United States
(12) Patent Application Publication
(10) Pub. No.: US 2010/0198123 A1
(43) Pub. Date: Aug. 5, 2010

(54) Wrap-around support sling

(76) Inventor: Kimberly Seff, Los Angeles, CA (US)

Correspondence Address: OBER / KALER;C/O ROYAL W. CRAIG, 120 EAST BALTIMORE STREET, SUITE 800, BALTIMORE, MD, 21202, US

(21) Appl. No.: 12/381,314

(22) Filed: Mar. 10, 2009

Related U.S. Application Data
(60) Provisional application No. 61/068,705, filed on Mar. 10, 2008.

(51) Int. Cl. A61F 5/00

(52) U.S. Cl. 602/4

(57) ABSTRACT

The present invention is a wrap-around support sling which comprises a reinforced elongate fabric strap that wraps around the patient's upper torso (over one shoulder and under the other arm) and is secured thereabout in a loop by hook and loop fasteners. The sling continues down the back and is wrapped around the patient's leg, where it is secured in a loop around the leg by hook and loop fasteners. The sling gives full body support, and may be gripped in both hands by an attending via integral handles positioned at the sternum and lumbar region to readily lift the patient out of a chair to a standing position without stress or risk of injury to either the patient or attending. The sling is rugged and possesses a simple and scalable design and may be economically manufactured and sold to provide for widespread use and disposability.

### CROSS-REFERENCE TO RELATED APPLICATION(S)

The present application derives priority from U.S. provisional application Ser. No. 61/068,705 filed Mar. 10, 2008.

### BACKGROUND OF THE INVENTION

1. Field of the Invention

The present invention relates to a wrap-around sling, and more particularly to a wrap-around sling adapted for sitting support.

2. Description of the Background

The elderly, disabled, and other individuals suffering from limited mobility often have great difficulty standing from a chair. It is usually necessary for attending nurses or family members to manually assist these disabled individuals to stand up, however this can lead to disabling back injuries for the attendants and can also risk pain or harm to the individual being assisted. Alternately, an adaptive seating system may be utilized to assist an occupant to achieve a sit-to-stand position. A number of prior efforts have been made to assist in this regard including the following.

United States Patent Application 20030192118 by Carbonneau et al. published Oct. 16, 2003 shows a Marpet Sling which attaches over both arms and legs, and is adapted to be hooked to a lifting device, including a ceiling hoist, for lifting and transferring a patient.

United States Patent Application 20060080775 by McWattie et al. published Apr. 20, 2006 and United States Patent Application 20050283907 by Dalton published Dec. 29, 2005 both show a lifting sling for supporting a person from a hoist via a full-back support.

Although the prior art support slings that assist an occupant to achieve a sit-to-stand position, as well as others have furthered technological development they all require cumbersome electromechanical lifts to raise an occupant from the sit-to-stand position.

Thus, it would be greatly advantageous to provide a wrap-around sling adapted to help an attending person securely support a patient from a sitting-to-standing position, the sling easily attaching by wrapping around the neck/torso and one leg by hook and loop fasteners, and providing integral point handles for a two-handed stand-up assist from the attending.

### SUMMARY OF THE INVENTION

It is, therefore, the primary object of the present invention to provide a wrap-around support sling which provides a convenient two-handed stand-up solution designed for full-body hoisting-support of a disabled patient from a sitting-to-standing position.

Another object of the present invention is to provide a wrap-around support sling that is fabricated of rugged materials providing an appropriate degree of flexibility, resiliency, durability, and longevity.

Still another object of the present invention is to provide a wrap-around support sling that can be quickly and easily secured around an individual in a seated position.

Yet another object of the present invention to provide a wrap-around sling that can be folded and easily stowed away for storage and/or transport.

Another object of the present invention is to provide a wrap-around support sling that possesses a simple and scalable design (i.e. it may vary in size to fit various body types).

An additional object of the present invention is to provide a wrap-around support sling that may be economically manufactured and sold to provide for widespread use and disposability.

These and other objects are accomplished by a wrap-around sling that provides a convenient two-handed stand-up assistive transfer solution for the elderly or disabled. The sling comprises a reinforced elongate fabric strap that wraps around the disabled person's torso (over one shoulder and under the other arm) and is secured thereabout in a loop by hook and loop fasteners. The sling continues down the back and is wrapped around the disabled person's leg, where it is likewise secured in a loop around the leg by hook and loop fasteners. The attached sling gives full body support, and may be gripped in both hands by an attending via integral handles positioned at the sternum and lumbar region to readily lift the disabled person out of a chair to a standing position without stress or risk of injury to either the disabled individual or attending. The support sling is rugged and possesses a simple and scalable design and may be economically manufactured and sold to provide for widespread use and disposability.

### BRIEF DESCRIPTION OF THE DRAWINGS

Other objects, features, and advantages of the present invention will become more apparent from the following detailed description of the preferred embodiments and certain modifications thereof when taken together with the accompanying drawings in which:

FIG. 1 is a front view of the sling 1 wrapped around a patient 6.

FIG. 2 is a rear view of the sling 1 partially wrapped around a patient 6.

FIG. 3 is a front view of the sling 1 looped underneath a disabled 6 individual's arm.

FIG. 4 is a front view of attendant 5 wrapping the sling 1 around patients' 6 leg.

FIG. 5 is a front view of the attendant 5 grabbing handle 7 of sling 1.

FIG. 6 is a front view of the attendant 5 grabbing handle 18 of sling 1.

FIG. 7 is a front view of the attendant 5 readily pulling handles 7, 18 to raise individual 6 up from the sitting position to standing securely.

### DETAILED DESCRIPTION OF THE PREFERRED EMBODIMENT

The present invention is a wrap-around sling 1 that serves as an assistive transfer device to ease the job of attending caregivers 5 in hoisting patients from a sitting to a standing position, and vice versa, as they are being transferred from bed to a commode, tub, wheelchair or the like.

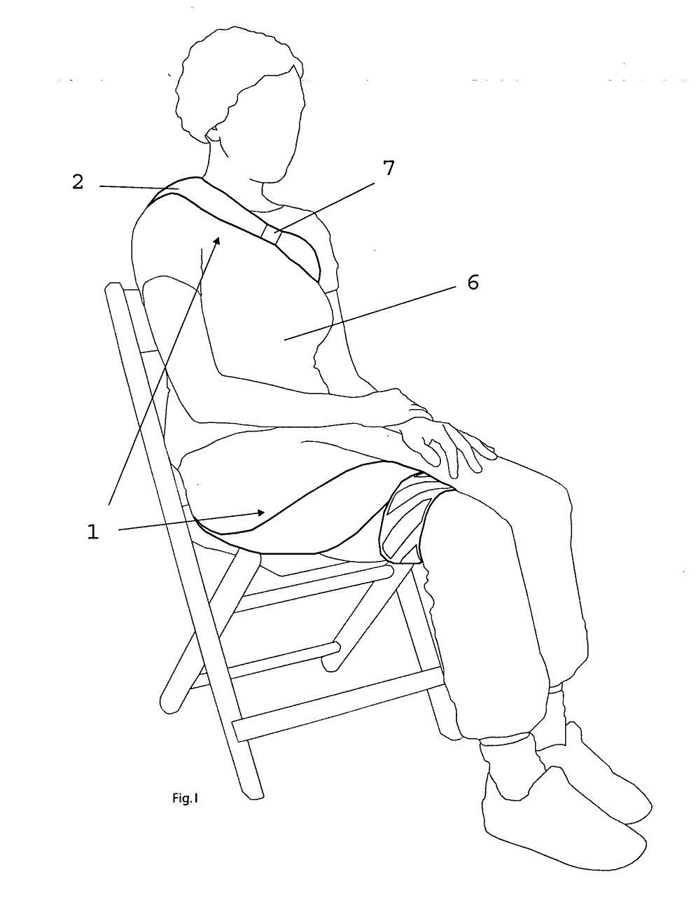

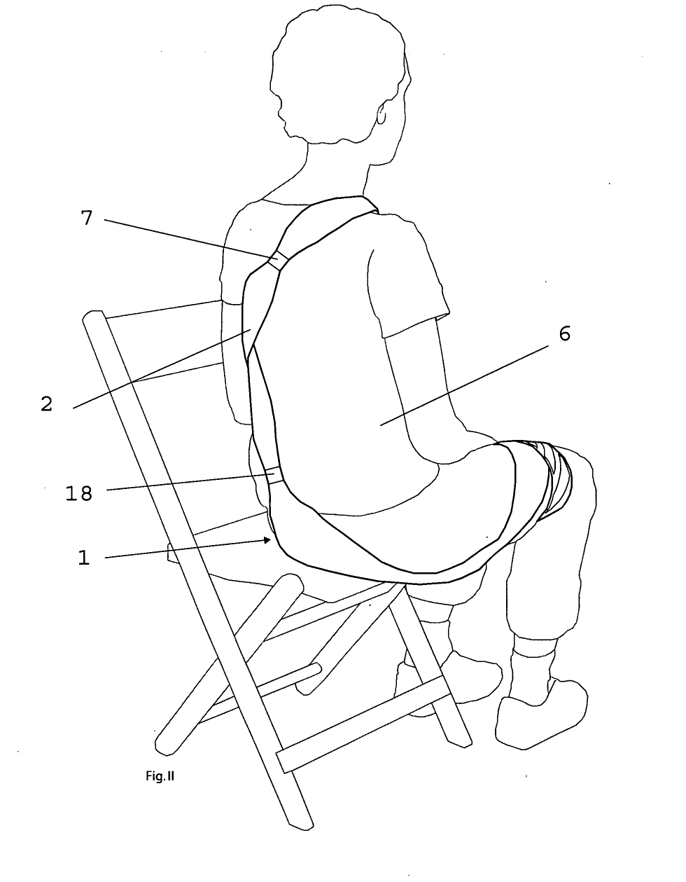

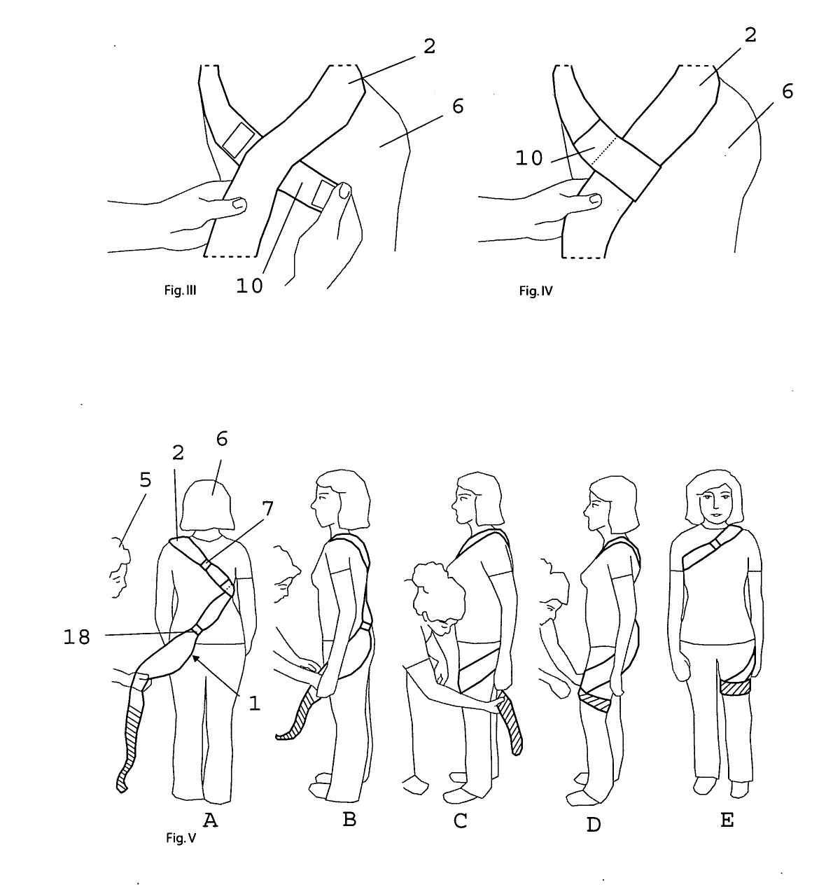

FIGS. 1 and 2 are front and rear views of the sling 1 wrapped and partially wrapped around a patient 6, respectively. The wrap-around support sling 1 is specifically suited for the elderly, disabled, and other individuals 6 suffering from limited mobility and is designed for a direct stand-up assist without help of any overhead supporting infrastructure such as a boom or hoist. It should be understood that OSHA requirements mandate the use of a boom or hoist for lifting patients in excess of 225 pounds, and so the present invention should not be used in excess of the 225 pound weight limitation.

The sling generally comprises a double-layer reinforced elongate fabric strap 2 that attaches to the upper torso by wrapping over one shoulder and under the other arm, extending frontally down the sternum, under an arm, around and down the back, and finally attaching to the opposing upper leg. The strap 2 bears two integral handles 7, 18 which allow an attending person to grip at the sternum and abdomen and more easily hoist the patient from a sitting to standing position.

The strap 2 generally comprises two elongate strips of fabric 8, 9 stitched together by a cross-stitch pattern for added ruggedness. The strips 8, 9 are generally patterned, cut, and sewn together, and then a piece of border fabric is sewn around the periphery. Both ends of the sling 1 are designed to loop around and attach into themselves by particular Velcro™ fasteners.

Elongate strap 2 is preferably comprised of a rugged material such as canvas, leather, suede, or other durable material to withstand numerous uses and the forces exerted from pulling an individual wrapped within sling 1. The strap 2 can come in a wide array of styles, sizes, and colors to appeal to individual taste. The strap 2 is preferably approximately 5 to 6 feet in length and approximately 6 inches in width, although the dimensions of the strap 2 may vary to fit various heights and body type.

FIG. 3 is a front view of upper end of strap 2 looped around the patient's neck and secured thereto by an overlay-type Velcro™ fastener. Specifically, the end 10 of the elongate strap 2 is bifurcated beyond handle 17 into an overhead length of strap 3, and an underlying length of strap 4 (obscured). The bifurcated strap ends 3, 4 are approximately 5 to 6 inches in length, and approximately 3 inches in width. It is preferable to cross-stitch 17 one end 14 of strap 3 and both ends 15 of straps 4 to elongate strap 2 for reinforcement and ruggedness. Both the overhead length of strap 3 and underlying length of strap are equipped with Velcro™ pads on their inner faces. Opposing Velcro™ pads are sewn onto the strap 2 midlength so that end of strap may be looped around the neck, and fastened onto itself at the Velcro™-equipped midlength by sandwiching the Velcro-laden midlength between the overhead length of strap 3 and underlying length of strap.

The sling 1 further comprises at least two handles 7, 18 formed of braided strips of Nylon sewn at both ends lengthwise along the strap 2, the sewing pattern being a box-pattern for reinforcement. The handles 7, 18 are positioned at spaced distances from strap 3. Preferably, each handle 7, 18 is positioned approximately 1-2 feet apart from the other such that when the sling 1 is wrapped around the disabled individuals' 6 body, one handle 7 is positioned on one of individual's 6 shoulders and the other handle 18 is positioned around the individual's 6 lumbar region. The handles 7, 18 allow an attendant 5 to lift an individual 6 wrapped around in the sling 1.

FIG. 4 is a front view of attendant 5 wrapping the sling 1 around patient 6. As seen in FIG. 4, the lower end of strap 2 also comprises opposing hook and loop fasteners at an end 11. The hook and loop fasteners 16 are adhered to the strap 2 to retain the sling 1 wrapped around the patient's leg. The hook and loop fasteners 16 allow an attendant 5 to secure the straps 2, 3 by exerting minimal pressure.

In use of the sling 1, the attendant 5 (nurse, family member, etc.) wraps the sling 1 around disabled individual 6 while the patient 6 is standing. The sling 1 is wrapped around the disabled individual's 6 upper torso (over one shoulder and under the opposite arm), and the transverse strap 3 is securely looped around the opposing end 11 of strap 2 via the hook and loop fasteners. The sling 1 continues down the back and the attendant 5 wraps it around the patient's 6 leg where it is likewise secured in a loop(s) around the disabled individual's 6 leg by the hook and loop fasteners on the strap 2. The attendant 5 can easily adjust the sling 1 around the patient's 6 leg and tighten or loosen the strap 2 around the by connecting the strap 2, via the hook and loop fasteners, to itself for added security or comfort. After securing the sling 1 around the patient 6, the patient 6 may move about without the sling 1 hindering arm or leg movements.

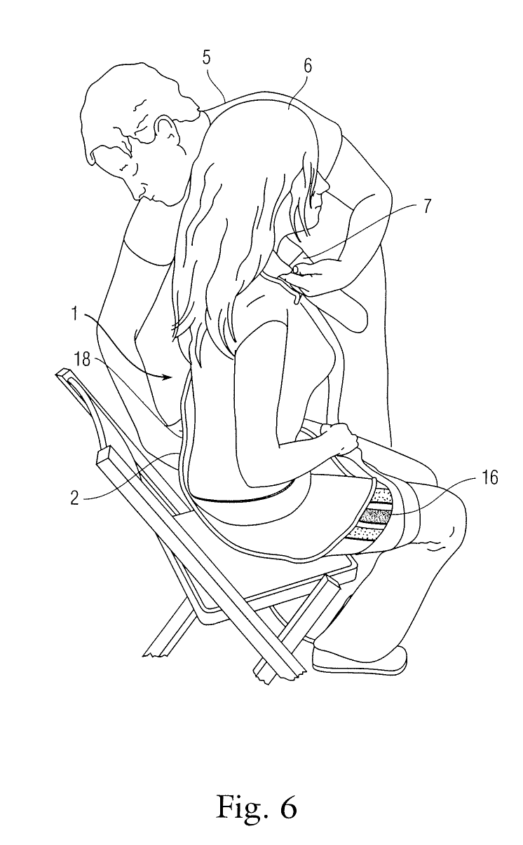

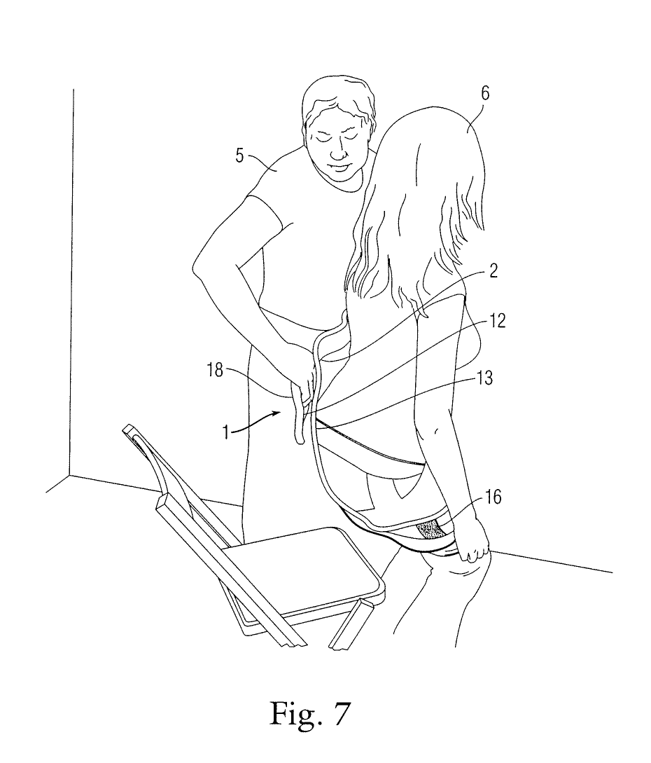

When sitting down and desiring to stand up, an attendant 5 may assist the individual 6 by pulling up on the sling. FIGS. 5 and 6 show the attendant 5 grabbing each handle 7, 18, respectively. With one hand the attendant 5 grabs the handle 7 at the sternum of patient 6 and with the other hand grabs the other handle 18 at the lumbar region of the patient 6. FIG. 7 shows the attendant 5 readily pulling the handles 7, 18 to raise individual 6 up from the sitting position to standing securely, without stress or risk of injury to either the patient 6 or the attendant 5. The sitting-to-standing action of the sling is an important feature. Initially, the same supporting dynamics of the chair are assumed by the sling 1, and so the patient remains cradled. The attending person exerts a lifting force via handle 18 rising upward against the buttocks of the patient, and a forward pull force via handle 7 to depart the chair. The patient's legs are completely unfrettered and so they easily rise from sitting to standing position. Meanwhile, the attending person gains the benefit of handles 7, 18 and no longer needs to stoop over so that the patient can reach over their shoulders. This significantly reduces the risk off back strain any the attendings.

The sling 1 is lightweight and portable and can be carried by nurses and attendings as an accessory. In a matter of seconds, the wrap-around support sling 1 can be applied to a patient and secured. This wrap-around support sling 1 efficiently allows an attendant 5 to assist an occupant in moving from the sit-to-stand position without an overhead hoist or support arm, and alleviates numerous problems arising from such assistance. Therefore, the rugged wrap-around support sling 1 of the present invention allows patients 6, with the help of their caregivers 5, a simple and effective means to sit-up with confidence and comfort.

As is evident from the above disclosure, the wrap-around support sling 1 of the present invention possess a simple, yet scalable design that may be economically manufactured and sold to provide for widespread use. The support sling 1 utilizes rugged materials chosen to provide the appropriate degree of flexibility, resiliency, durability, longevity required by the nature of their usage, and yet their cost is minimal to allow ready disposal.

Having now fully set forth the preferred embodiment and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth in the appended claims.

I claim:

1. A wrap-around support sling comprising:
an elongate fabric strap; and
a plurality of handles attached to said strap,
whereby said strap wraps around a patient's upper torso over one shoulder and under opposite arm and continues down said patient's back and is wrapped around said person's leg providing a two-handed sit-to-stand solution for attendants caring for said patient.
